(19) United States
(12) Patent Application Publication
(10) Pub. No.: US 2011/0301492 A1
(43) Pub. Date: Dec. 8, 2011

(54) MULTI-CHANNEL IMPEDANCE CARDIOGRAPHY AND METHOD OF MULTI-CHANNEL IMPEDANCE CARDIOGRAPHY

(75) Inventor: Jürgen Lamp, Harju (EE)

(73) Assignee: JR MEDICAL OU, Harju (EE)

(21) Appl. No.: 13/208,839

(22) Filed: Aug. 12, 2011

Related U.S. Application Data
(63) Continuation-in-part of application No. PCT/EE2010/000005, filed on Feb. 12, 2010.

(30) Foreign Application Priority Data
Feb. 12, 2009 (EE) U2009000014

(51) Int. Cl. A61B 5/053

(52) U.S. Cl. 600/547

(57) ABSTRACT

Multi-channel impedance cardiograph comprises a sine generator, a multiplexer, a high-pass filter, an amplifier, an analogue-code converter and a microcontroller. A compensation circuit reduces the required order of the analogue-code converter. The compensation circuit comprises a second sine signal generator, an adder, a comparator for comparing its input signal with a reference signal, and a counter. Both sine signal generators are synchronised and the signal from the body is compensated by the signal of the second sine signal to normalize the input signal of the analogue-code converter. The first sine signal generator and the counter are started simultaneously. The counter stops when the comparator's output reverses polarity. The phase shift between the signals of first and second sine signal generators is calculated from the counter content. The amplitude of the compensation signal generator is adjusted so that the output code of the analogue code converter is within working range.

### RELATED APPLICATIONS

This application is a continuation-in-part of PCT Appl. No PCT/EE2010/000005—filing date Feb. 12, 2010 which claims priority to Estonian Appl. No U2009000014—filing date Feb. 12, 2009 which are herein incorporated by reference for all purposes.

### TECHNICAL FIELD

The rising popularity of the bioimpedance method as an easy, cheap and non-invasive measuring method forces the developers to pay more attention to the development of multifunctional devices. By implementing the bioimpedance measuring method over the whole body we can get information about the cardiovascular system, breathing function and the balance of extracellular liquid. The more channels are used, the more simultaneous information is received about the blood supply of different organs. Impedance cardiographs with up to two channels are produced industrially for measuring such important parameters as stroke volume (SV), cardiac output (CO) and pulse wave velocity (PWV). In connection with widening technical possibilities it is possible to develop impedance cardiographs with more than two channels, which also could be used for investigation of segmental blood supply.

### BACKGROUND ART

Multi-channel impedance cardiograph is described in U.S. Pat. No. 4,807,638, where the second channel is used to measure pulse wave velocity (PWV). The shortcoming is that the whole signal received from thoracic part is used as a starting point during PWV measurement. 2-channel impedance cardiograph is also described in U.S. Pat. No. 6,228,033.

More than 2 channels are in the device described in Finnish patent FI 105773 B, where current and voltage measuring electrodes are commutated to get information about the cardiothoracic part.

The circuit for measuring blood supply of extremities is described in patent application WO 98/53737, where a multiplexer is also used. An analogous solution is also contained in US 2004/0171961. A multi-channel bioimpedance measuring circuit is also described in patent application US 2005/0177062.

A schematic diagram of multi-channel impedance cardiograph is also described in patent application WO 2005/010640 (FIG. 5). The device consists of a multiplexer that commutates the current and voltage electrodes attached to different segments of the patient's body according to the microcontroller's program. There are an amplifier, high-pass filter, amplifier with gain-control and analogue-code converter in the multiplexer's output. The digitised signal is processed with microcontroller. The signal is commutated with the help of a divisor and a switch so that in multiplier it is multiplied by itself (measurement of active component R) or sine signal corrected with phase (measurement of impedance Z), derived from the sine table and phase corrector. Then the result is added up in the adder, entered in the memory, transformed to be proportional to resistance R and transmitted to the low-pass filter and subtractor to subtract the ΔR component from R. As a result basal resistance Rb and alternating component ΔR are acquired.

As the authors of the described invention point out, the required measuring accuracy for segmental and whole body impedance measurement is 10⁻⁵-10⁻⁶, which results in the order of the ADC converter of up to 20. If for example the pulse wave velocity from the distal parts of hands or legs is measured, the measurement accuracy will be 10⁻⁷, which results in an ADC order of 23. Using an ADC with such high order makes the measuring scheme complicated as 3-byte data will be used and there will be problems with guaranteeing the signal-noise ratio required for using younger bits.

In the known solution a low-pass filter is used to separate ΔR from R, which unavoidably introduces a time constant. The recommended cut-off frequency of the low-pass filter is <0.7 pulse frequency. At a heart rate of 60 beats per minute the time constant introduced by the low-pass filter would be ˜0.16 s. By introducing such time constant the frequency of channel commutation is reduced significantly. Usually a 200 Hz measuring frequency is used for commutation of physiological signals to transfer the signal without distortions, especially in case of electrocardiographic signals.

### SUMMARY OF INVENTION

The object of the invention is a measuring circuit for a multichannel impedance cardiograph that allows using lower-order ADC compared to known solutions and where the commutating frequency is not reduced by a low-pass filter. This goal is achieved by providing a compensation circuit between an output of the multiplexer and the input of the analogue-code converter. The compensation circuit compensates the amplitude of the output signal of the multiplexer with a compensation signal with the appropriate amplitude and phase, so that a standardised signal is transmitted to the analogue-code converter input. The advantage of the invention is the absence of low-pass filter that would reduce the channel commutation speed, and the possibility to use lower-order analogue-code converter ADC as most of the static component has been separated from the whole signal previously.

### BRIEF DESCRIPTION OF DRAWINGS

FIG. 1 shows the block diagram of the device according to one embodiment of the invention.

FIG. 2 shows the measurement of phase shift F between DDS1 and comparator signals.

FIG. 3 shows the measurement of amplitude, where A is maximum amplitude, B is compensation amplitude and 0 marks zero line.

FIG. 4 shows the ADC input signal in case of 6 channels (impedance channels K1 to K6).

FIG. 5 shows the placement of electrodes in case of 6-channel measurement.

FIG. 6 shows measured signals: ECG-ECG signal, AA -aortic arch, LA-left arm, LL-left leg, RA-right arm, RL-right leg, WB-whole body.

Explanation of abbreviations used in figures:

DDS1, DDS2-synchronised digital synthesisers,

U/I-voltage/current converter,

MUX-analogue multiplexer,

HPF-high-pass filter,

A1, A2-amplifiers,

SUM-adder,

ADC-analogue-code converter,

COMP-comparator,

MPU-microcontroller,

PC-computer,

ECG-ECG amplifier.

REF-reference input signal for the comparator.

### DETAILED DESCRIPTION

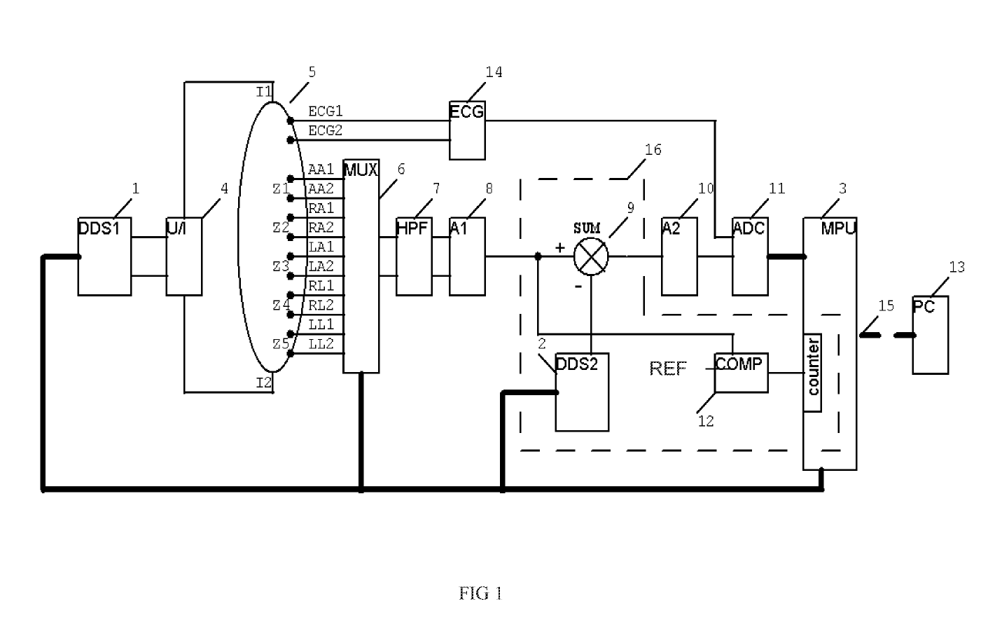

A device according to one embodiment of the invention is shown in FIG. 1. To achieve multi-channels two sine signal generators, a so-called DDS (Direct Digital Synthesiser) 1 and 2, controlled by a microcontroller 3, are used. Appropriate DDS is for example analogue device AD9958 by Analog Devices. This contains two independent sine generators which may be mutually synchronised.

Generally radio frequency 30-100 kHz is used to measure bioimpedance components associated with heart function and breathing. Higher frequencies are used to measure intracellular structure. The presented solution allows the use of different measurement frequencies. DDS1 output voltage is converted to current with voltage-current converter 4 and relayed usually to the distal body parts of the patient 5 with electrodes I1 and I2.

Electrode pairs AA1-AA2, RA1-RA2, LA1-LA2, RL1-RL2 and LL1-LL2, connected by a cable with an analogue multiplexer 6, are used to get segmental signals. The commutated signal passes a high-pass filter 7, which separates unwanted low-frequency noise and also an electrocardiographic signal component. Further the signal is amplified in an amplifier 8 and directed to the (+) input of an adder 9.

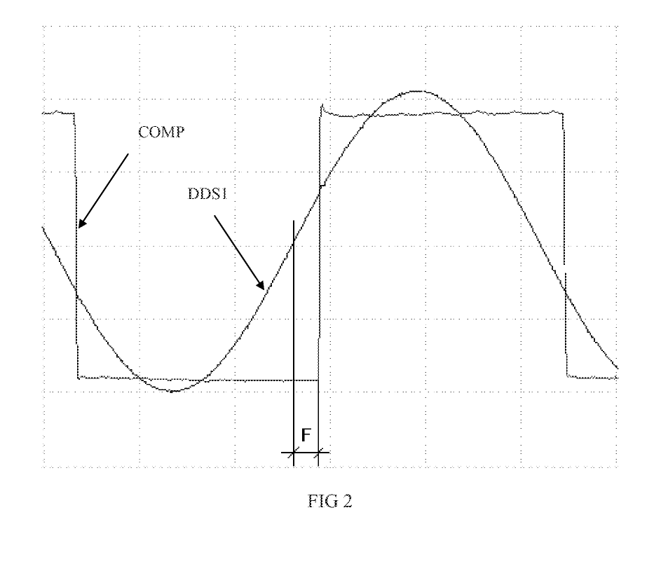

The first step is the measurement of a phase shift F of a compensation signal is carried out as follows (see FIG. 2). Simultaneously with the activation of DDS1 a counter inside microcontroller is activated with the microcontroller's clock rate. The counter is stopped when the DDS1 sine signal passes comparator's 12 reference (REF) value and the front of the comparator 12 changes to positive. Comparator's 12 output turns positive when amplifier's A1 output passes the REF value. Measurement of phase shift Fn is done for each channel separately and Fn values are memorized by microcomputer 3. Phase shift Fn between DDS1 and output of an amplifier A1 is used later for the compensation of each channel. Usually the comparator's reference (REF) value is 0 V. The obtained number of impulses by counter is proportional to phase shift Fn.

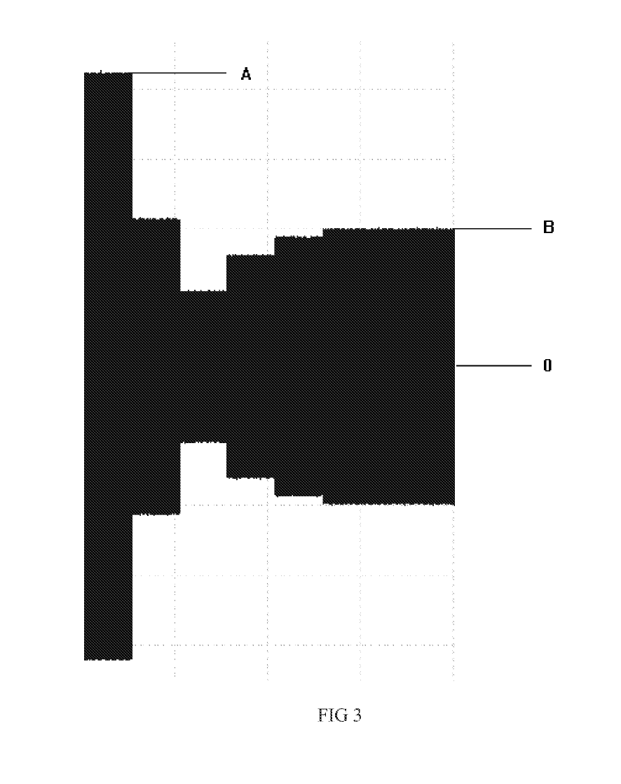

Next step is to measure compensation amplitude. The compensation signal from DDS2 (see FIG. 3) with a certain phase shift Fn for each channel with maximum amplitude is initially directed to the (−) input of the adder. The obtained difference is amplified with an amplifier 10 and its amplitude value is measured with a fast-acting sampling analogue-code converter 11. The timing of ADC is controlled by a microcontroller 3. The gain of amplifier A2 determines by what order the measuring accuracy of ADC may be reduced. ADCs with a conversion time of less than approximately 1 to 3 microseconds are suitable. The required ADC conversion time depends on the frequency of the current given to the patient. If this frequency is lower than 100 kHz, which is typical of impedance cardiographs, a conversion time less than 1 to 3 microseconds is sufficient. Measurement takes place at the sine peak at T/4 where T is a period of radiofrequency current. As there is initially a big signal difference in the inputs of the adder 9, an overflow code will be obtained from the ADC 11. Then the compensation signal is reduced twofold. If the signal polarity in the ADC input does not change, the compensation signal is again reduced twofold. If now the polarity changes, the compensation signal is increased by ¼, etc., until the signal in the input of the ADC 11 is in the pre-determined range (working range). The standardisation of the ADC 11 input signal takes place with a 1-2-4-8 algorithm, where each subsequent step is one-half the previous and the direction is determined by the ADC overflow sign.

With the circuit shown in FIG. 1 the alternating component ΔZ is subtracted from the whole of signal Z without using a low-pass filter. By doing this the whole signal frequency spectrum starting from the direct component is obtained.

Formula (1) is used to calculate Z₀:

Z₀=[(K+a*N/M)−b]/a (ohm) (1)

where K is the balancing amplitude of DDS2,

N is the amplitude of the signal in ADC 11 input,

M is calibration coefficient. M is usually the variation of ADC 11 code corresponding to a 1-ohm variation in multiplexer input,

a and b are coefficients depending on circuit parameters.

ΔZ is calculated with formula (2):

ΔZ=N/M (ohm) (2)

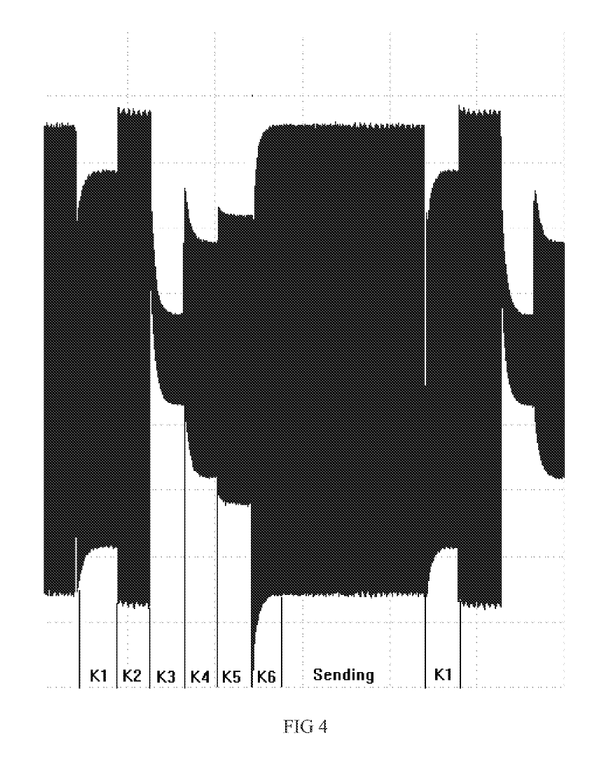

The multi-channel operation of the device is as follows: at first initial phase shifts Fn of all channels and compensation signal amplitudes are measured with method described above, then the channels are commutated with a measuring frequency (see FIG. 4) so that for each channel a corresponding compensation signal amplitude and phase shift are used to drive DDS2. A difference between measured signal and compensation signal emerges in the output of the adder 9, which is then amplified before transmission to the ADC. Sine signal packages are form in the ADC 11 input, one package corresponding to each channel. The duration of the package depends on the measuring frequency and number of channels. FIG. 4 shows the oscillogram of the 6-channel impedance cardiograph in the ADC 11 input. The visible distortions during channel commutation develop because of the removal of a polarization potential between skin and electrodes with an high-pass filter 7 and therefore the initial part of the package cannot be used for measuring. So-called “sending” package, during which calculations are done and information is transmitted to the computer, is also contained in one measuring cycle.

A measuring frequency up to 200 Hz is sufficient to reproduce physiological signals in the computer.

Usually the impedance cardiographs also have a channel ECG 14 for measuring the electrocardiographic signal ECG which is used for algorithm synchronisation. In the ADC 11 input the required commutation between the impedance signal and ECG signal takes place.

Data from the ADC is transmitted to the computer 13 via a cable or wireless connection (such as WiFi or Bluetooth™, etc) 15. Further processing of initial data and reporting of results takes place in the computer.

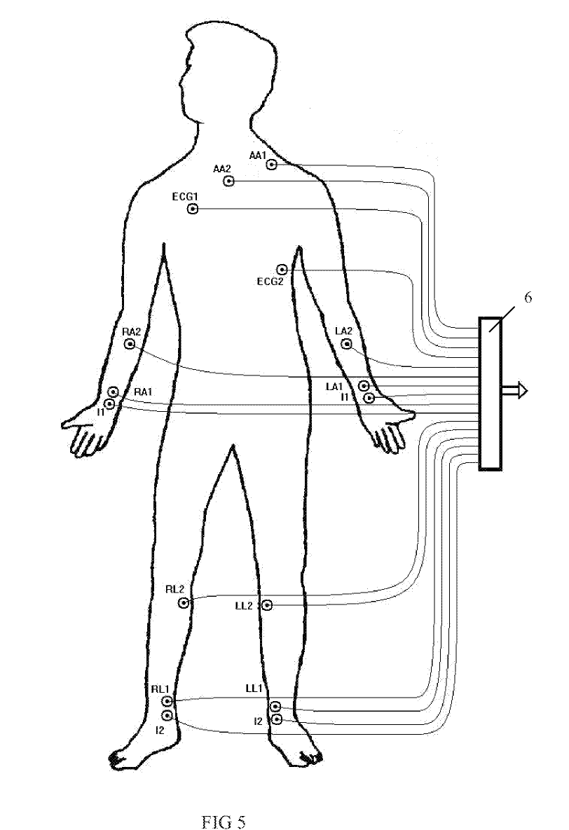

FIG. 5 shows the placement of electrodes in case of 6-channel measurement. Electrodes I1 and I2 are used for feeding current. On the aortic arch the signal is obtained from electrode pair AA1-AA2, which guarantees higher accuracy of pulse wave starting point determination compared to known solutions (e.g. electrode positions proposed in U.S. Pat. No. 4,807,638 and methods described by Kööbi T, Kähonen M, Iivainen T, Turjanmaa V. on paper Simultaneous non-invasive assessment of arterial stiffness and haemodynamics—a validation study. Clin Physiol Funct Imaging. 2003 January; 23(1):31-6). Signals are obtained from legs from electrode pairs RL1-RL2 and LL1-LL2 and from arms correspondingly from RA1-RA2 and LA1-LA2. The whole body signal (WB) is measured between electrode pairs RL1-LL1 and RA1-LA1. For this RL1 is connected to LL1 and RA1 to RL1 in the multiplexer for the duration of WB channel commutation. Such electrode placement allows simultaneous measurement of pulse wave velocity PWV from the aortic arch to extremities, whereby at the same time also cardiac stroke volume (SV) and cardiac output (CO) and other haemodynamic indicators are measured in WB channel.

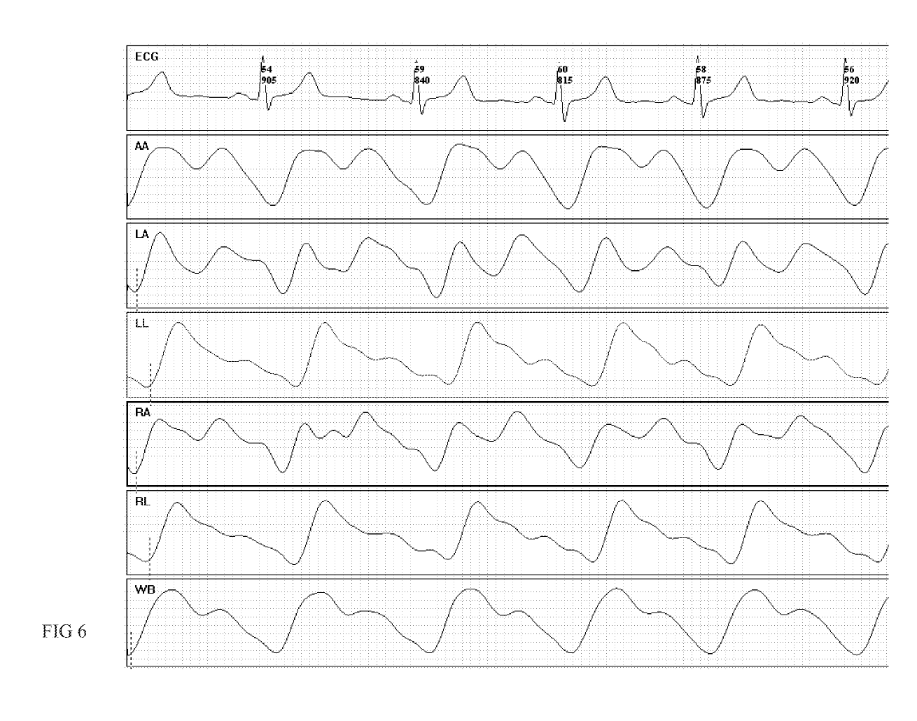

FIG. 6 shows signal graphs, where ECG-ECG signal and impedance signals are AA-aortic arch, LA-left arm, LL-left leg, RA-right arm, RL-right leg, WB-whole body, correspondingly.

In addition to pulse wave velocity (PWV) it is possible to calculate the indicators characterising the blood supply of extremities like pulse volume PV and minute volume F from segmental LA, LL, RA and RL impedance signals, using analogous methodology as for cardiac stroke volume (SV) and cardiac output (CO) (see U.S. Pat. No. 6,228,033 which is herein incorporated by reference).

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A multi-channel impedance cardiograph, comprising:
a first sine generator, wherein an output of the first sine generator is connectable to a pair of current electrodes attachable to a patient's body;
a multiplexer having a plurality of inputs, wherein each input of the multiplexer is connectable to a one of plurality of voltage electrodes attachable to different segments of the patient's body and an output of the multiplexer is connected to an input of the adder where the alternating component of impedance is subtracted from the whole impedance;
an analogue-code converter for transforming an analog signal to digital signal code;
a microcontroller, which is programmed to control the operation of the multiplexer so that the inputs of the multiplexer are successively connected to the output of the multiplexer, and to control of subtraction of the alternating component of impedance from the whole impedance; and
a compensation circuit, connected between the output of the multiplexer and the input of the analogue-code converter, wherein the compensation circuit comprises a second sine signal generator, an adder, a comparator and a counter, wherein a second input of the adder and an input of the comparator are connected to the output of the multiplexer, wherein the microcontroller is programmed to start the first sine signal generator and the counter simultaneously and to stop the counter when the output of the comparator has reversed its polarity, and a phase shift between the the first sine signal generator and multiplexer output is determined by the counter content, wherein a phase of the second sine signal generator is corrected according to obtained phase shift and an amplitude is corrected iteratively until the output code of the analogue-code converter has changed from an overflow code to a code within a working range, and alternating component of the impedance is fed from the output of the adder through an amplifier to the analogue-code converter.

2. A device according to claim 1, wherein a phase shift and the amplitude compensation codes corresponding to alternating current signals obtained from each pair of voltage electrodes are saved in a memory of the microcontroller.

3. A device according to claim 2, wherein the microcontroller is programmed to commutate the multiplexer with a measuring frequency and to change the amplitude and phase of the second sine generator according to the counter reading and pre-determined algorithm and the obtained amplitudes of sine packages have been measured with the fast-acting analogue-code converter and saved in the memory of the microcontroller.

4. A device according to claim 3, wherein the output of the microcontroller is connected to a computer over a cable or a wireless connection and the microcontroller is adapted for data saving, processing, presenting and reporting of results.

5. A device according to claim 4, wherein the microcontroller is programmed to calculate the channel basal impedance Z₀ on the basis of the second sine generator compensation amplitude and analogue-code converter output code according to formula Z₀=[(K+a* N/M)−b]/a (ohm), where K is the balancing amplitude of the second sine generator, N is the output code of the analogue-code converter, M is the calibration coefficient, and a and b are coefficients depending on the circuit parameters.

6. A device according to claim 5, wherein the microcontroller is programmed to calculate the impedance signal ΔZ on the basis of the analogue-code converter output code using the formula ΔZ=N/M (ohm).

7. A method for multi-channel impedance cardiography in a system comprising a first sine generator, connected to a pair of current electrodes attached to a patient's body, an analogue code converter, having an input connected to at least one pair of voltage electrodes, attached to a patient's body, a comparator, wherein a first input of the comparator is connected to a reference signal and a second input of the comparator is connected with the input of the analogue code converter, a counter and a microprocessor for controlling the operations of the system, the method comprising:
starting both said first sine wave generator and said counter at the same time, thereby introducing a first sine wave current into the patient's body through said pair of current electrodes;
receiving a response voltage from the body through said at least one pair of voltage electrodes;
inputting said response voltage to an input of said comparator;
stopping the counter at the moment when the output of comparator reverses its polarity, and
determining the phase shift between the first sine wave and the response sine wave from the reading of the counter.

8. A method as in claim 7, comprising generating a second sine wave using a second sine wave generator, said second sine wave being delayed compared to said first sine wave by said phase shift, and subtracting said second wave from said response voltage to form a standardized input signal for said analogue-code converter to keep the output code of the analogue-code converter within a working range.

9. A method as in claim 8, wherein an amplitude of said second sine wave is adjusted according to the code of the analogue-code converter so that to replace an overflow code in the analogue-code converter output with a code within a working range.

10. A method according to claim 9, comprising determining said phase shift and said amplitude for a plurality of channels, each channel having its own pair of voltage electrodes, by subsequently switching each channel to a input of a analogue-code converter and storing said phase shift and said amplitude for each channel in a memory of a microprocessor, and measuring said impedance for each channel, using said phase shifts and amplitudes for each channel by consequently switching from one channel to the next
